(19) United States
(12) Patent Application Publication
(10) Pub. No.: US 2024/0090914 A1
(43) Pub. Date: Mar. 21, 2024

(54) PIEZOELECTRIC SURGICAL DEVICE AND METHODS OF PERFORMING SPINAL SURGERY

(71) Applicant: Ra'Kerry Rahman MD PA, Houston, TX (US)

(72) Inventor: Ra'Kerry Kahlil Rahman, Houston, TX (US)

(21) Appl. No.: 17/945,678

(22) Filed: Sep. 15, 2022

(51) Int. Cl. A61B 17/32; A61B 17/02; A61B 34/30; A61B 90/30

(52) U.S. Cl. CPC A61B 17/320068; A61B 17/0218; A61B 34/30; A61B 90/30; A61B 2017/32007; A61B 2017/320072; A61B 2090/306; A61B 2090/373; A61B 2217/007

(57) ABSTRACT

A surgical device providing a body defining a interior volume, a surgical tip configured to impart a vibration to a contacted object and a piezoelectric arrangement configured to initiate a piezoelectric effect to actuate a vibration in the surgical tip, wherein the surgical tip is configured to be inserted endoscopically into a patient.

### CROSS-REFERENCE TO RELATED APPLICATIONS

None.

### FIELD OF THE DISCLOSURE

Aspects of the disclosure relate to surgical devices. More specifically, aspects of the disclosure relate to a piezoelectric device for use in spine surgery. In some non-limiting aspects of the disclosure, piezoelectric devices used in spine surgery are coupled with robotic devices to accomplish accurate surgical procedures, with enhanced patient recovery times.

### BACKGROUND

Spinal surgery can be a complex, expensive and intrusive method for various patients that require correction of a defect or injury. Due to the complex nature of the spine, practitioners in this field must be highly trained in order to perform such complex tasks.

As necessitated by this type of surgery, networks of nerves, blood vessels, cartilage and bone are encountered on a regular basis by surgeons. Conventional apparatus and methods to perform surgery require use of manual manipulation of tissue and bone through cutting and retraction elements. There are many drawbacks to use of such conventional apparatus. Conventional apparatus have a limit upon how much tissue and bone can be severed. This limitation requires surgeons to make various passes of cutting and retraction, increasing the overall time for the surgery. This increases the cost of such surgery.

There are also drawbacks for patient care using conventional apparatus and methods. Prolonged surgery requires a patient to be sedated for longer periods of time. Moreover, the incisions that are made are relatively large, requiring more post-operative care.

As will be understood, certain spinal surgery can be a delicate procedure requiring great care in performing required operations. While requiring a very accurate procedure, bones in the spine, for example, can be very hard, requiring great force in order to perform needed surgical techniques. Thus, the surgeon is required to exert large amounts of force during the operation, but be delicate in certain aspects to prevent undue trauma to the patient. Such trauma can include excessive blood loss, excessive incision size, potential for nerve damage arising from the surgical procedure and potential for infection among other issues. There is a need to minimize such trauma to patients in an effort to enhance surgical results. There is a further need to provide enhanced recovery times associated with minimally invasive procedures.

Surgical spinal operations often occur in classes or typical types of surgeries. For example, some surgical operations require placement of a post which requires mechanical attachment of a post into the spinal column. As this type of procedure is common, there is a need in the industry to allow for consistency in conducting such procedures. Conventional operations do not provide for such repetition.

While general physiology of a person is known, each patient is different thereby causing some variation for the attending physician. Identifying critical structures during spinal surgery would help the physician in preventing inadvertent contact with sensitive structures. There is a need to equip different surgical devices with sensors such that the attending physician may quickly and accurately identify structures close to surgical areas. There is also a further need to provide for three-dimensional control of surgical devices in spinal surgery to aid in placement of surgical devices.

Conventional apparatus and methods only provide for manual operation of tools related to piezoelectric surgery. In the case of spinal surgery, such operations may be physically strenuous upon the surgeon. There is a need, therefore, to provide the opportunity for a surgeon to conduct a surgery in an accurate fashion without the physical exertion required by conventional apparatus.

There is also a need to provide apparatus and methods that are easier for surgeons to operate than conventional apparatus and methods.

There is a further need to provide apparatus and methods that do not have the drawbacks discussed above.

There is a still further need to reduce economic costs associated with operations and apparatus described above with conventional tools.

There is also a still further need to be able to cut tissue and bone more efficiently than conventional apparatus.

There is also a further need to be able to make cutting of tissue and bone more selective wherein large incisions are unnecessary.

There is also a need for an apparatus that will allow spinal surgeons to enhance productivity without the need for expensive medical training.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are therefore not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a surgical device is described. The surgical device may comprise a body defining a interior volume, wherein the body has a first end and a second end, wherein the first end and the second end are hollow. The surgical device may also comprise a surgical tip configured to impart a vibration to a contacted object. The surgical device may also comprise a piezoelectric arrangement configured to initiate a piezoelectric effect to actuate a vibration in the surgical tip, wherein the surgical tip is configured to be supported by the body and be inserted through the body endoscopically into a patient and wherein the piezoelectric arrangement is connected to the surgical tip to impart the vibration to the surgical tip and wherein the piezoelectric arrangement and surgical tip are configured to be placed into the body to a surgical site within the patient.

In another embodiment, a surgical device is disclosed. The surgical device may comprise: a body defining an interior volume, wherein the body has at least a first end and a second end, wherein the first end and the second end are hollow and at least one optical fiber configured to illuminate an area inside a patient near the second end of the body. The surgical device may also comprise a surgical tip configured to fit through the first end and the second end of the body and into a patient in an endoscopic incision to a spinal area to impart a vibration to a contacted object within the patient. The surgical device may also comprise a piezoelectric arrangement configured to initiate a piezoelectric effect to actuate a vibration in the surgical tip and at least one optical fiber to receive video images near the surgical tip. The surgical device may also comprise at least one display device connected to the optical fiber such that a surgeon may visually see actions of the surgical tip during operation. The surgical device may also comprise a sterile irrigation system configured to impart a fluid to an area near the surgical tip during operation and a suction system configured to remove at least one of fluid and objects from the endoscopic incision.

In another embodiment, a method is disclosed. The method may comprise making an incision into a patient spinal area through a robotic surgical device and inserting an endoscopic piezoelectric device into the incision with the robotic surgical device. The method may also comprise performing at least one function using a piezoelectric effect with the piezoelectric device on the patient with the robotic surgical device; and closing the incision with the robotic surgical device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted, however, that the appended drawings illustrate only typical embodiments of this disclosure and are therefore not to be considered limiting of its scope, for the disclosure may admit to other equally effective embodiments.

FIG. 1 is schematic diagram of one example embodiment of the disclosure.

FIG. 2 is a plan view of a processing arrangement used in accordance with the aspects of FIG. 1.

FIG. 3 is a plan view of different metallic tips used for the device of FIG. 1.

FIG. 4 is a method of performing spinal surgery in accordance with one aspect of the disclosure.

FIG. 5 is a plan view of a robotic arm used in conjunction with piezoelectric spinal surgery.

FIG. 6 is a plan view of a control layout for the robotic arm used in conjunction with piezoelectric spinal surgery of FIG. 5.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood, however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and shall not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second” and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and\or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood, however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Aspects of the disclosure relate to methods and apparatus for conducting spinal surgery with a piezo-electrical endoscopic device. To date, piezo-electrical devices are used in many fields, such as engineering maintenance activities, loudspeakers and various consumer goods. Piezo-electric surgery is known, such as that described in U.S. Pat. No. 6,695,847. In this patent, a piezo-electric device is used to perform rudimentary and surficial types of surgery. Such apparatus and methods, however, are inappropriate for endoscopic surgery as the surgeon must control a hand gripped body to make surface based cuts.

Aspects of the present disclosure allow for more precise cutting needed for surgery by providing an endoscopic surgical ability not previously available. In one aspect, the device 100 has a body 102. The body 102 has a first end 104 and a second end 106. The body 102 may be configured as a tube such that the first end 104 and the second end 106 are open. In embodiments, materials, such as optical fibers 108, and cutting arrangements 110 may be transmitted through the body 102 first end 104 to the second end 106. At the second end 106, the cutting arrangement 110 may protrude such that a cutting capability is provided to a surgeon. The diameter of the body 102 may be 5.5 millimeters. This diameter allows for small incisions to be made into a body, such as at a spine, with pinpoint accuracy, compared to larger incisions needed with conventional apparatus. The optical fibers 108 may be provided with the capability to provide a surgeon the ability to see the progress of the surgery from a close position. To help with clarity of the pictures obtained, the optical fibers 108 may be provided with an optical enhancement device, such as a lens, to obtain optical images to be passed through the optical fiber. Images may be sent to a processing system 112 that may process the data for display. A monitor 114 may be used to enhance and/or enlarge the optical data/images obtained by the optical fibers.

To aid in obtaining illuminated picture capability for incisions within the body of a patient, certain portions of the optical fibers 108 are configured to distribute light to the second end 106 of the body 102. The amount of light may be varied by the surgeon to minimize or maximize the light transmitted through the fiber optics so that overexposure or underexposure is minimized. The amount of light may be controlled by a lighting system 116 that is connected to the processing system to illuminate the optical fibers 108.

As a further aid in surgery, a suction apparatus 120 may be located either within the body 102 or attached to an outside diameter of the body 102. The suction apparatus 120 may remove fluids or objects that may obstruct the surgeons view of the procedure. The suction from the suction apparatus 120 may be varied through use of a variable speed pump that may be controlled by the surgeon or an assistant during the operation.

In embodiments, the cutting arrangement 110 is driven by a piezoelectric arrangement 130 to allow the surgeon control over various parameters. For example, the cutting arrangement 110 may be fine-tuned such that only bone is cut by the cutting arrangement. The fine tuning may be done through a control panel 132. Such a setting allows the surgeon the ability to perform the cutting portion of the surgery more quickly as unintended cuts of tissues that are not to be disturbed during the surgery are left alone. As the piezo-electric driven cutting arrangement 110 may be tuned, the cutting arrangement 110 may be adapted such that only ligaments are cut, if such a capability is necessary. In a similar fashion, cutting may be adapted to only specific types of soft tissue.

In an example surgery, an assistant or the surgeon may selectively choose between different cutting modes to limit the amount of disturbance for the patient. Thus, the surgeon may selectively choose different structures to be cut maximizing efficiency and minimizing disturbance.

Piezoelectric arrangement 130 allowing for the generation of cutting by the cutting arrangement 110 may be located within a housing 135. The housing 135 may be configured in any type of shape necessary to control the cutting capabilities of the cutting arrangement 110. Aspects provide for a power supply 140 that provides electrical current at a voltage to a piezo-electric control panel 132. In embodiments, a high frequency vibration is generated by the control panel 132 such that a metallic tip 190 of the cutting arrangement 110 is vibrated at a frequency between 25 kHz and 35 kHz, as non-limiting values. As will be understood, bone density may vary according to the type of bone encountered during surgery. Frequency may be varied by the surgeon such that individuals with a more dense bone structure can be handled by the surgeon. Equally important, is that some bone structure may be less dense than other. To this end, a patient with a thinner bone structure may undergo cutting at an acceptable rate for the surgeon by varying the frequency according to the needs of the patient.

In other embodiments, a sterile irrigation system 160 may be included within the body 102 to allow the surgeon to irrigate an area that is to be cut or that is being cut, to allow the suction apparatus 120 the capability to remove materials from within or near the incision. The sterile irrigation system 160 may be configured such that a surgeon can actively start the system 160 upon a need. In further embodiments, the irrigation system 160 may be configured to activate at specific times, such as during cutting. A controller 162 may be used to control the irrigation system 160. The controller 162 may be configured to actuate a pump placed within the irrigation system 160.

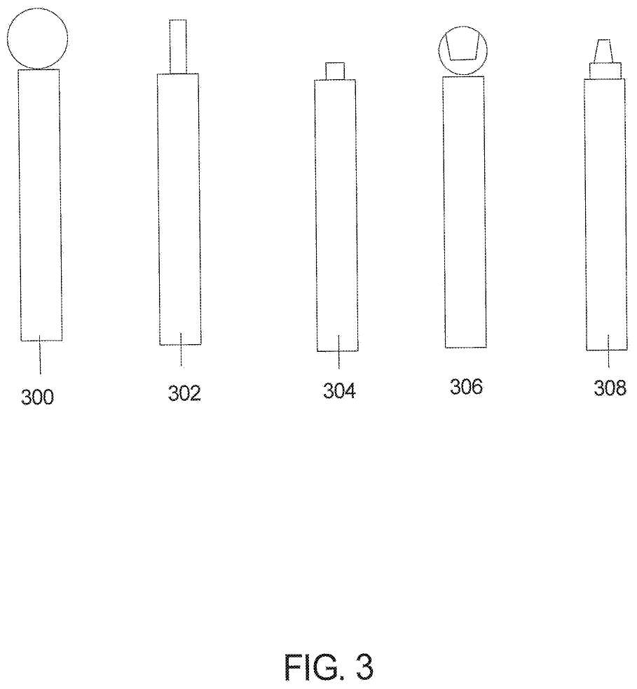

The cutting arrangement 110 is minimized compared to conventional apparatus to allow for small incisions to be made and/or worked within. The cutting apparatus has a metallic tip 190 that is made of a stainless steel such that is vibrated by the piezoelectric arrangement 130. The metallic tip 190 may have different shapes according to the needs of the surgeon, the rate of cutting desired, the density of the bone encountered, the size of the cutting, angularity of the cut desired and other factors. Referring to FIG. 3, a round tip 300, a flat tip 302, a square tip 304, a rounded scraper 306 and flat chisel tip 308 are illustrated and each one may be used as the metallic tip 190. Each of the descriptive tips referred to in FIG. 3 are merely illustrative and therefore variations from the illustrated embodiment are possible. In other embodiments, more complex shapes may be used to allow for incisions to be made. For example, two intersecting lines forming a 90 degree corner may be used. Such a configuration would allow, for example, two incisions to be made at one time, rather than requiring the surgeon to make two separate incision lines. Such incisions would increase the overall efficiency of the operation. Other types of complex or two-dimensional cutting shapes may be provided, including a 45 degree angle, a 30 degree angle, a 60 degree angle and cross-shape. In addition to more complex shapes, the actual tips or probe parts may be configured to allow for the surgeon to use the tips more efficiently. For example, each of the tips may be angled to a greater or lesser degree according to the reach needs of the surgeon. The tips may also have bearing surfaces that are hardened to increase the longevity of the tip under surgical conditions. Such hardening may be through annealing, tempering or creating a carbide surface, such as diamond, on the outside of the tip. The tips are made of metallic materials to allow for hardness when cutting tissue or bone. The metallic materials may also be cleaned to provide for a sterile instrument after use as the metallic materials do not absorb liquid or trap liquid. In other embodiments, the tips may be serrated, if necessary, to aid in the cutting of materials. The tips may be cleaned, in a non-limiting embodiment, within an autoclave or disinfectant. In embodiments, the tips may be inserted through a cavity opening or pathway that penetrates into the body. This pathway may be created to allow the piezoelectric tips to be inserted into the body where the tips may be used. The pathway may also be incorporated with an irrigation canal to allow for a flushing of fluid to be accomplished to aid in visual identification of structures.

In embodiments, the pathway may also be equipped with sensors to allow the attending physician to identify localized body structures. For example, electrical resistors may be placed at the end of the pathway to read a localized potential electrical current in the pathway. Presence of such a resistance in the circuit may indicate the proximity of a nerve within the body. As will be understood, various types of electrical monitoring may be performed, including resistance, impedance and current flow.

In conventional apparatus, cutting efficiency is dependent upon the pressure placed upon the object being cut. In the instance of cutting bone, a conventional surgical saw is only as efficient as the pressure placed by the surgeon between the bone and the saw. Without pressure, cutting is not achieved. There is a need, therefore, for a surgeon using a conventional saw to force the saw or burr into areas needing cutting. Aspects of the disclosure, however, do not rely on pressure. Mere contact with the high frequency vibration of the tip can result in cutting. In aspects, a gripping surface 179 is provided for the device 100 to allow the surgeon to grip the device 100. The gripping surface 179 may be through a pen grasping motion. An off and on switch 181 may be placed on the gripping surface 179 to allow the doctor to start and/or stop the vibration.

As sensitive structures may be encountered during surgery, forces of less than 1 N may be exerted to achieve cutting rates sufficient for surgery. At the evidence of heat being generated through the cutting, irrigation may be added through the sterile irrigation system, thereby minimizing heat damage to tissue and/or bone during surgery. As will be understood by surgeons, cortical bone, the hardened outside surface of bone structures, may be more difficult to cut than interior bone and/or structures. In embodiments, a first frequency may be chosen by a surgeon to cut the cortical bone, while a second frequency may be chosen by the surgeon to cut inner structures or non-cortical bone.

In embodiments, the piezoelectric surgical device 100 allows for not only cutting of bone, but also of other structures, such as structures formed by calcinosis. In these embodiments, calcium depositions interspersed with tissues can present difficulty for surgeons because of the varying matrix of materials to be cut. The use of a piezoelectric surgical device 100 can alleviate such concerns and remove or cut only what is needed for treatment of the patient by selectively choosing a frequency specially suited for removing a specific type of structure. The piezoelectric surgical device 100 may be configured to provide a pathway into a patient or may be used in conjunction with other devices to create a pathway into the patient. Such devices may have separate openings for irrigation, optics and other equipment, as needed.

A specific advantage with use of a small piezoelectric surgical device 100 is that the size of the device 100 may be advantageously used in surgery that uses specific types of equipment. As an example, compared to an “open back” spine surgery, aspects of the disclosure may be used in conjunction with other types of surgical equipment, such as a tubular retractor. As such common place equipment may be used in conjunction with aspects of the disclosure, economic costs are decreased for surgeons who use or upgrade to a piezoelectric surgical device 100 as equipment previously purchased may still be used and the need for specialized equipment is minimized. In embodiments, aspects of the piezoelectric surgical device 100 can be used in conjunction with surgery for herniated disks, fusion of disks, laminectomies, transforaminal lumbar interbody fusion, facetectomy, foraminotomy and discectomy. In embodiments, due to the function of the piezoelectric surgical device, temperature monitoring may be accomplished on the device 100 or through a supplementary arrangement. If temperatures reach a pre-designated threshold, the irrigation system 160 may be actuated to cool the temperatures inside the incision. As will be understood, the irrigation system 160 may be equipped with its own temperature system that identifies the temperature of water being removed from the incision. The irrigation system 160 may also be configured with coolers to refrigerate the water being delivered the patient and further cool body structures undergoing or in close proximity to the device 100.

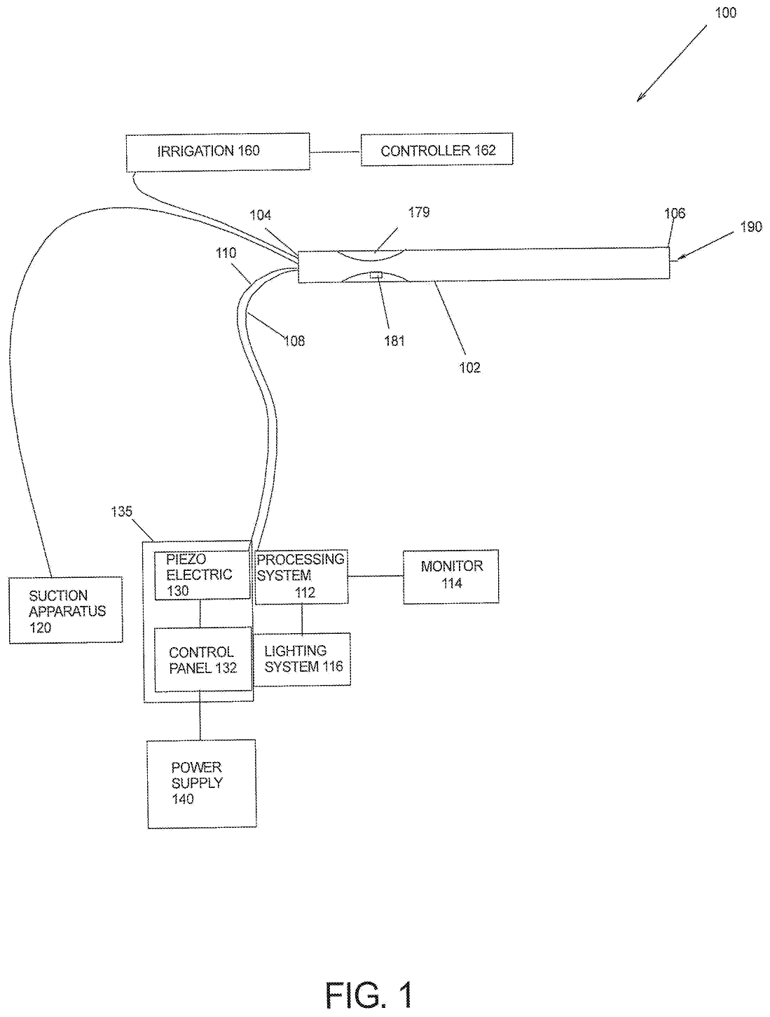

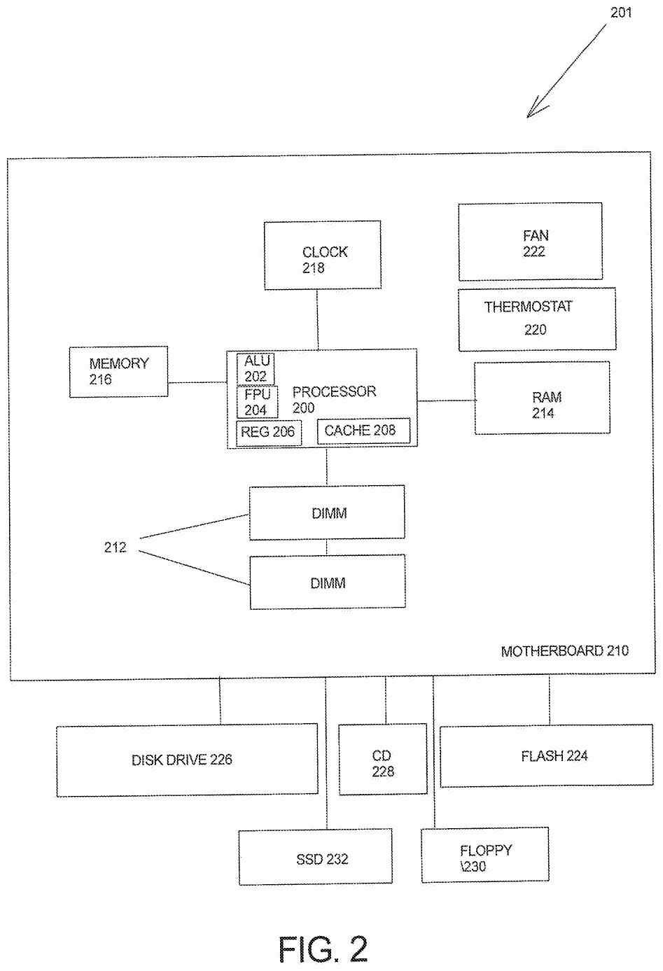

Referring to FIG. 2, a computing apparatus 201 used in the control of equipment of FIG. 1 is illustrated. In FIG. 2, a processor 200 is provided to perform computational analysis for instructions provided. The instruction provided, code, may be written to achieve the desired goal and the processor may access the instructions. In other embodiments, the instructions may be provided directly to the processor 200. The processing accomplished by the processor 200 may control many aspects or aid in controlling the functions of the irrigation system 160, the controller 162, the control panel 132, the lighting system 116, the processing system 112 and the suction apparatus 120.

In other embodiments, other components may be substituted for generalized processors. These specifically designed components, known as application specific integrated circuits (“ASICs”) are specially designed to perform the desired task. As such, the ASIC's generally have a smaller footprint than generalized computer processors. The ASIC's, when used in embodiments of the disclosure, may use field programmable gate array technology, that allow a user to make variations in computing, as necessary. Thus, the methods described herein are not specifically held to a precise embodiment, rather alterations of the programming may be achieved through these configurations.

In embodiments, when equipped with a processor 200, the processor may have arithmetic logic unit (“ALU”) 202, a floating point unit (“FPU”) 204, registers 206 and a single or multiple layer cache 208. The arithmetic logic unit 202 may perform arithmetic functions as well as logic functions. The floating point unit 204 may be a math coprocessor or numeric coprocessor to manipulate number more efficiently and quickly than other types of circuits. The registers 206 are configured to store data that will be used by the processor during calculations and supply operands to the arithmetic unit and store the result of operations. The single or multiple layer caches 208 are provided as a storehouse for data to help in calculation speed by preventing the processor 200 from continually accessing random access memory (“RAM”).

Aspects of the disclosure provide for the use of a single processor 200. Other embodiments of the disclosure allow the use of more than a single processor. Such configurations may be called a multi-core processor where different functions are conducted by different processors to aid in calculation speed. In embodiments, when different processors are used, calculations may be performed simultaneously by different processors, a process known as parallel processing.

The processor 200 may be located on a motherboard 210. The motherboard 210 is a printed circuit board that incorporates the processor 200 as well as other components helpful in processing, such as memory modules (“DIMMS”) 212, random access memory 214, read only memory, non-volatile memory chips 216, a clock generator 218 that keeps components in synchronization, as well as connectors for connecting other components to the motherboard 210. The motherboard 210 may have different sizes according to the needs of the computer architect. To this end, the different sizes, known as form factors, may vary in sizes from a cellular telephone size to a desktop personal computer size. The motherboard 210 may also provide other services to aid in functioning of the processor, such as cooling capacity. Cooling capacity may include a thermometer 220 and temperature controlled fan 222 that conveys cooling air over the motherboard 210 to reduce temperature.

Data stored for execution by the processor 200 may be stored in several locations, including the random access memory 214, read only memory, flash memory 224, computer hard disk drives 226, compact disks 228, floppy disks 230 and solid state drives 232. For booting purposes, data may be stored in an integrated chip called an EEPROM, that is accessed during start-up of the processor. The data, known as a Basic Input/Output System (“BIOS”), contains, in some example embodiments, an operating system that controls both internal and peripheral components.

Different components may be added to the motherboard or may be connected to the motherboard to enhance processing. Examples of such connections of peripheral components may be video input/output sockets, storage configurations (such as hard disks, solid state disks, or access to cloud-based storage), printer communication ports, enhanced video processors, additional random access memory and network cards.

The processor and motherboard may be provided in a discrete form factor, such as personal computer, cellular telephone, tablet, personal digital assistant or other component. The processor and motherboard may be connected to other such similar computing arrangement in networked form. Data may be exchanged between different sections of the network to enhance desired outputs. The network may be a public computing network or may be a secured network where only authorized users or devices may be allowed access.

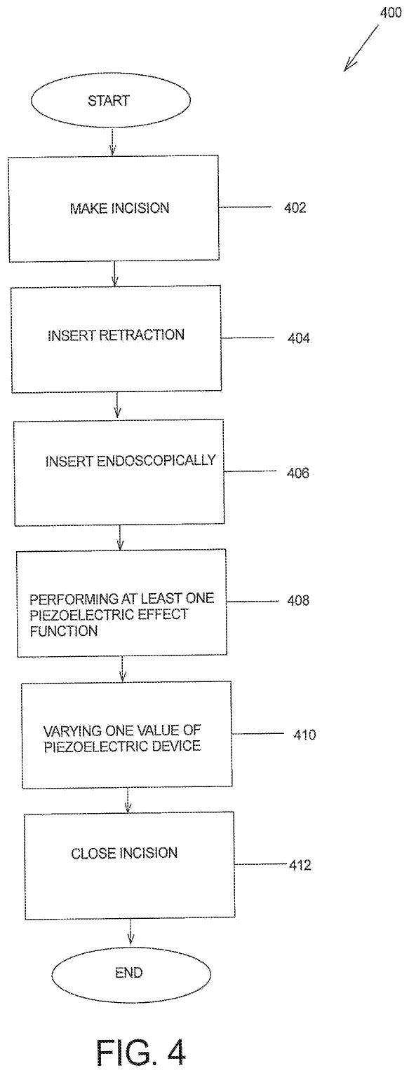

Referring to FIG. 4, a method 400 for performing endoscopic surgery is illustrated. The method 400 may include, at 402, making an incision into a patient spinal area. At 404, the method may include inserting a retraction device into the incision in the patient spinal area prior to inserting the endoscopic piezoelectric device into the incision. At 406, the method may also include inserting an endoscopic piezoelectric device into the incision. At 408, the method may include performing at least one function using a piezoelectric effect with the piezoelectric device on the patient. At 410, the method may also include varying at least one value of the piezoelectric device for the at least one function. At 412, the method may also include closing the incision.

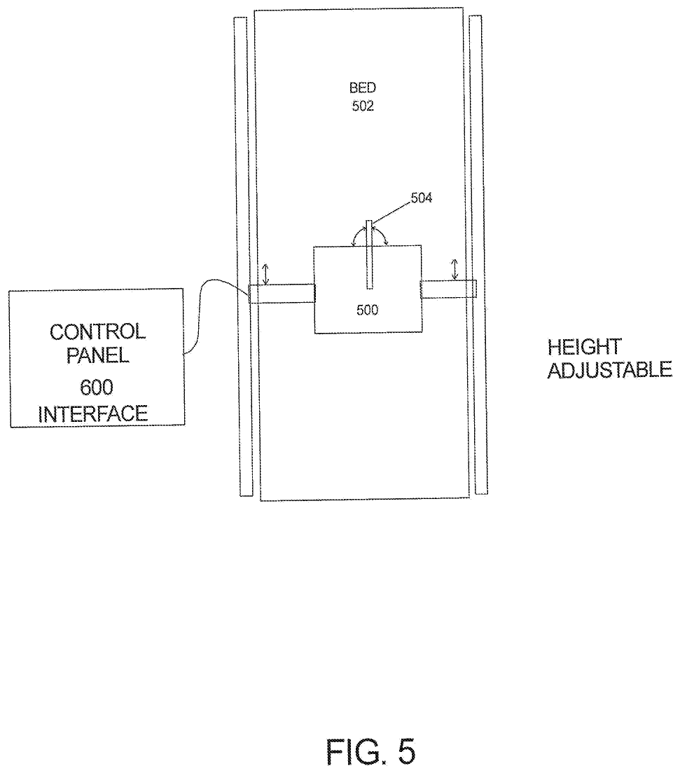

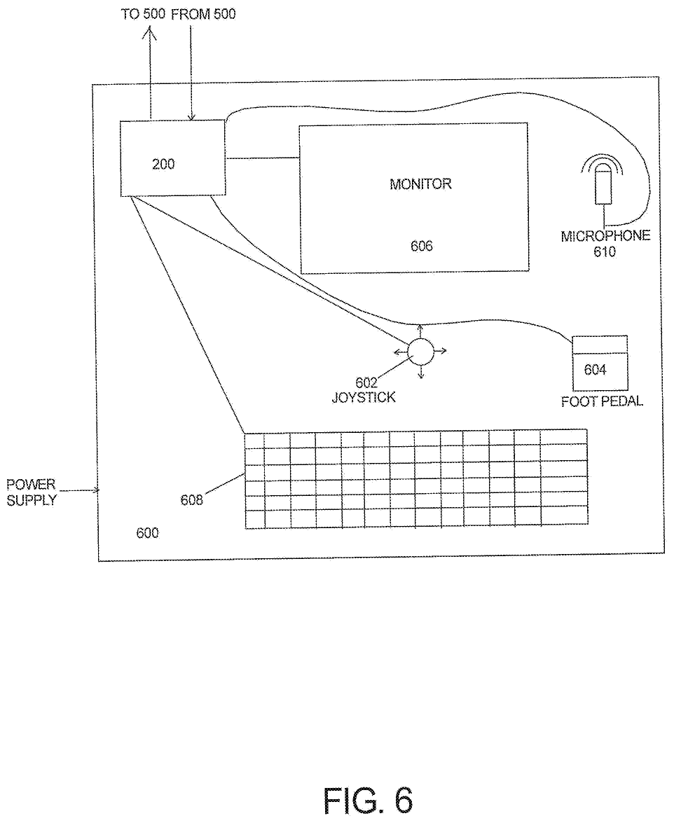

Referring to FIG. 5, a surgical robotic device 500 utilizing a piezoelectric surgical device is illustrated. The surgical robotic device 500 is equipped to perform robotic surgery on a patient. In one example embodiment, a patient may be loaded onto a table 502 that supports the patient during a medical procedure. The surgical robotic device 500 may be affixed to the table/bed 502 to provide a “known” position in three-dimensional space upon which to perform the procedure. The surgical robotic device 500 may be controlled through an interface 600, as described in relation to FIG. 6. The surgical robotic device 500 is connected to a power supply to provide uninterruptable power. In embodiments, the components of FIG. 2 may be used to control the movements of the devices of FIG. 1 and FIG. 5. In one embodiment, the surgical robotic device 500 is powered by both electrical input from an electrical service as well as a battery back-up. If the electrical service is interrupted during the medical procedure, the electrical supply may automatically be switched to a battery back-up supply. In embodiments, precision scanning of a patient, such as a CT scan or MRI scan may be used in establishing a correct positioning of the patient prior to surgery. This precision scanning may establish three-dimensional reference for control of the surgical robotic device 500 used in surgery.

In embodiments, the surgical robotic device 500 may include a robotic arm 504 that positions needed components and performs the surgical operation. As described previously, a piezoelectric device 100 may be used and inserted into the pathway. The surgical robotic device 500 may also include capabilities to create the initial incision for entry of the pathway. Data from the instruments used in the surgical procedure may be fed to a physician who controls the surgical robotic device 500. As such the surgeon may be remotely located from the patient but still perform the necessary operation. In this instance, a surgeon may schedule multiple surgeries for a single day, vastly increasing the overall efficiency of medical operations.

In some embodiments, the surgical robotic device 500 may be configured with auxiliary lights, magnification optics and irrigation operations. Other optional components may include suction capabilities. Other capabilities may include ability to link to other computers for provision of a video or video snapshot. Recording of surgical activities may be accomplished by on-board video equipment and storage devices. The surgical robotic device 500 may also be configured to independently monitor respiration, heart rate, oxygen saturation levels and other patient vital signs. In other embodiments, a communications link may be provided to the surgical robotic device 500 where independent equipment may feed data into the surgical robotic device 500 for display.

Controls of the surgical robotic device 500 may be through an autonomous logic and programming. In other embodiments, the surgical robotic device 500 may allow an attending physician to not only view the procedure, but also control a robotic arm, for example, for incising, drilling and other surgical functions. Referring to FIG. 6, controls for the surgical robotic device 500 of FIG. 5, are illustrated. An interface 600 is provided with a joystick 602 that controls horizontal motion of the robotic arm. Vertical control may be provided through a connected foot pedal 604 or other similar device. Actions at the surgical sight may be displayed through a monitor 606. A computer arrangement may display data from a scan of a patient for the attending physician so surgical operations may be performed to a resolution of one (1) mm. The computer arrangement may be, for example, the embodiment provided in FIG. 2. In some embodiments, interlocks may be placed on movement of the surgical robotic device 500 to prevent hitting critical patient structures, providing a fail-safe design. As will be understood, an attending physician may override such interlocks, as necessary. A keyboard 608 may be provided for entering notes during the operation. A microphone 610 may be provided for connection for communication to provide instructions to the surgical robotic device 500 or to other surgeons.

In one example embodiment, a logical circuit may be used to automate the surgery being performed. Such automation may occur through a pre-programmed logic that interfaces with a logic controller. The logic controller may interface with the interface 600 as illustrated in FIG. 6. The logic controller may provide not only independent control, but also data to the surgeon in non-autonomous mode. For example, the surgeon may be provided with a “force feedback” to allow the surgeon to feel resistance during the surgery that the piezoelectric arrangements are experiencing. The amount of force feedback may be altered, at the discretion of the surgeon, to provide greater or lesser amounts of force. Such interface with the surgeon allows the surgeon to conduct a surgery without extending large amounts of physical effort, while still allowing the surgeon to “feel” the progress of the surgery. In this way, a surgeon may remotely conduct a surgery, but may be remote from the patient. In one example embodiment, force feedback may be accomplished through a rotating mass system hooked to a joystick, allowing vibrations to be sensed by the surgeon during the operation. Vibration may be increased or decreased in frequency according to the frequency of the piezoelectric tip or other function.

As will be understood, the pre-programmed logic may be altered for conducting operations for different types of piezoelectric surgery to allow adaptability of the design. With this in mind, the pre-programmed logic may alert a surgeon in non-autonomous mode, that certain critical bodily structures are close to the piezoelectric tip, requiring special care by the surgeon. For example, the pre-programmed logic may allow for alerting of the surgeon based upon a zone of influence around the tip. This zone of influence may be altered according to the precision of the surgeon. For example, a surgeon may create a zone of influence of approximately 1 mm for sensitive surgery where complicated structures are near the incision area. Greater or lesser amounts for the zone of influence may be chosen. Zones of influence may be set according to details of the patient or set to the potential accuracy of the surgical equipment. Such zones of influence may be set according to other parameters, such as surgical best practices or insurance best practices, in order to limit potential liability of the surgeon.

As will be understood in method steps described above, such steps may be stored in the random access memory, read only memory, flash memory, computer hard disk drives, compact disks, floppy disks and solid state drives.

Different input/output devices may be used in conjunction with the motherboard and processor. Input of data may be through a keyboard, voice, Universal Serial Bus (“USB”) device, mouse, pen, stylus, Firewire, video camera, light pen, joystick, trackball, scanner, bar code reader and touch screen. Output devices may include monitors, printers, headphones, plotters, televisions, speakers and projectors. In embodiments, visual representation may be made by a display device. Such display devices may be light emitting diode monitors, cathode ray monitors, liquid crystal displays or other similar devices.

Embodiments of the disclosure provide many advantages over conventional apparatus. The use of piezoelectric cutting provides greater efficiency than conventional rotary drill and saw operations. As the piezoelectric cutting capability is greater, a surgeon may become less tired and perform the surgical operation much quicker with less blood loss. Excessive incision size is minimized and the potential for nerve damage arising from the surgical procedure and potential for infection among other issues is also minimized.

Surgical spinal operations often occur in classes or typical types of surgeries. For example, some surgical operations require placement of a post which requires mechanical attachment of a post into the spinal column. As this type of procedure is common, there is a need in the industry to allow for consistency in conducting such procedures. Conventional operations do not provide for such repetition.

Aspects disclosed provide a patient the capability to allow for variation between patients. Aspects may help in identifying critical structures during spinal surgery preventing inadvertent contact with sensitive structures. When equipped with data from a scanning operation and a robotic surgical device, three-dimensional control is provided that is superior to conventional devices.

In some embodiments, error protection circuitry is provided. During surgical operations, a self-checking sequence may compare data obtained during surgery to a hypothetical data set. If the self-checking sequence indicates a discrepancy between the surgical data set and the hypothetical data set, an error may be indicated and provided to the surgeon. In instances, the surgery may be halted and the robotic arm and/or other equipment may be returned to a “fail safe” mode to protect a patient. In such instances, manual control may be provided back to the surgeon and the potential error provided to the surgeon to allow for surgeon corrective action.

In some embodiments, control input for the medical devices may also be done through three-dimensional analysis and display through, for example, a virtual reality headset. In this non-limiting embodiment, a medical scan, such as a CT scan or an MRI scan may produce data that may be visually represented to the surgeon. The surgeon may then use a sensored glove, pointing apparatus or other input device to pinpoint the area for drilling, treatment or other surgical activity. Recording of data from position recording apparatus attached to the surgical instruments or robotic arm may then pinpoint the surgical process. The self-checking sequence may then compare the pinpointed surgical process to pre-surgery scans taken to allow for visual depiction of the progress. Such visual depiction may be through three-dimensional representation, LED screen technology or visual depiction by camera. By staying within a predefined path of surgery, unwanted necrosis is avoided, presenting overall superior recovery times and reduced potential complications. Due to less tissue damage resulting from the piezoelectric surgery, edema is also minimized. Thus, the use of piezoelectric surgery allows for superior overall results compared to conventional osteotomes.

As will be understood, aspects of the robotic surgery system may be configured to handle different types of surgical components and/or in vivo components. Such components may include, pedicle screws, cages (such as lumbar cages and anterior interbody cages), dissectors, probes, forceps, elevators, spreaders, rongeurs, drills, drill bits.

In one example embodiment, a surgical device is disclosed. The surgical device may provide for a body defining an interior volume, wherein the body has at least a first end and a second end, wherein the first end and the second end are hollow. The surgical device may also comprise a surgical tip configured to impart a vibration to a contacted object. The surgical device may also comprise a piezoelectric arrangement configured to initiate a piezoelectric effect to actuate a vibration in the surgical tip, wherein the surgical tip is configured to be supported by the body and be inserted through the body endoscopically into a patient and wherein the piezoelectric arrangement is connected to the surgical tip to impart the vibration to the surgical tip and wherein the piezoelectric arrangement and surgical tip are configured to be placed into the body to a surgical site within the patient.

In another example embodiment, the surgical device may be configured wherein the piezoelectric arrangement is configured such that a user may select a vibration frequency of the arrangement.

In another example embodiment, the surgical device may further comprise at least one optical fiber configured to at least one of illuminate an area being operated upon and receive optical data from the area being operated upon and wherein the at least one optical fiber is configured to be inserted separately into the body and separately positionable within the patient.

In another example embodiment, the surgical device may further comprise at least one of a suction arrangement configured to remove debris from an area adjacent to the surgical tip, and an irrigation system configured to provide a flow of fluid to the patient, wherein each of the suction arrangement and the irrigation system are independently positionable within the patient

In another example embodiment, the surgical device may be configured wherein the suction arrangement is controlled by a user.

In another example embodiment, the surgical device may further comprise a sterile irrigation system configured to deliver a fluid to an area adjacent to the surgical tip wherein the irrigation system is independently positionable within the patient and wherein the irrigation system is inserted into the body through a separate opening of the body.

In another example embodiment, the surgical device may be configured wherein the sterile irrigation system is configured to be controlled by a user.

In another example embodiment, the surgical device may be configured wherein the tip is configured as one of a rounded tip, serrated, a two dimensional cutting profile, an angle, a chisel and a wedge.

In another example embodiment, a surgical device is described. The surgical device may comprise a body defining an interior volume, wherein the body has a first end and a second end, wherein the first end and the second end are hollow and at least one optical fiber configured to illuminate an area inside a patient neat the second end of the body. The surgical device may also comprise a surgical tip configured fit through the first end and the second end of the body and into a patient in an endoscopic incision to a spinal area to impart a vibration to a contacted object within the patient. The surgical device may also comprise a piezoelectric arrangement configured to initiate a piezoelectric effect to actuate a vibration in the surgical tip and at least one optical fiber to receive video images near the surgical tip. The surgical device may also comprise at least one display device connected to the optical fiber such that a surgeon may visually see actions of the surgical tip during operation, a sterile irrigation system configured to impart a fluid to an area near the surgical tip during operation and a suction system configured to remove at least one of fluid and objects from the endoscopic incision.

In another example embodiment, the surgical device may further comprise at least one recording device connected to the at least one display device to record activities during the operation.

In another example embodiment, the surgical device may be configured wherein at least one of the sterile irrigation system and the suction system are at least one of user controlled and computer controlled.

In another example embodiment, the surgical device may be configured wherein the piezoelectric arrangement is configured such that a user may select a vibration frequency of the arrangement.

In another example embodiment, the surgical device may be configured wherein the tip is configured to cut at least one of bone and soft tissue.

In another example embodiment, the surgical device may be configured wherein the tip is configured as one of a rounded tip, a hook, serrated, a chisel, a wedge and a burr.

In another example embodiment, the surgical device may be configured wherein the tip is configured to be placed in an incision less than 1 inch in length.

In another example embodiment, the surgical device may be configured, wherein the irrigation system is configured with a suction system configured to remove at least one of fluid and objects from the endoscopic incision.

In another example embodiment, a method is disclosed. The method may comprise making an incision into a patient spinal area, inserting an endoscopic piezoelectric device into the incision, performing at least one function using a piezoelectric effect with the piezoelectric device on the patient and closing the incision.

In another example embodiment, the method may further comprise varying at least one value of the piezoelectric device for the at least one function.

In another example embodiment, the method may be performed wherein the value is a frequency of vibration of a surgical tip of the piezoelectric device.

In another example embodiment, the method may be performed wherein the function is a cutting of bone with a surgical tip of the piezoelectric device.

In another example embodiment, the method may further comprise inserting a retraction device into the incision in the patient spinal area prior to inserting the endoscopic piezoelectric device into the incision.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A surgical device comprising:
a body defining a interior volume, wherein the body has at least a first end and a second end, wherein the first end and the second end are hollow;
a surgical tip configured to impart a vibration to a contacted object;
a piezoelectric arrangement configured to initiate a piezoelectric effect to actuate the vibration in the surgical tip, wherein the surgical tip is configured to be supported by the body and be inserted through the body endoscopically into a patient and wherein the piezoelectric arrangement is connected to the surgical tip to impart the vibration to the surgical tip, and wherein the piezoelectric arrangement and surgical tip are configured to be placed into the body to a surgical site within the patient.

2. The surgical device according to claim 1, wherein the piezoelectric arrangement is configured such that a user may select a vibration frequency of the arrangement.

3. The surgical device according to claim 1, further comprising:
at least one optical fiber configured to at least one of illuminate an area being operated upon and receive optical data from the area being operated upon, and wherein the at least one optical fiber is configured to be inserted separately into the body and separately positionable within the patient.

4. The surgical device according to claim 1, further comprising:
at least one of a suction arrangement configured to remove debris from an area adjacent to the surgical tip, and an irrigation system configured to provide a flow of fluid to the patient, wherein each of the suction arrangement and the irrigation system are independently positionable within the patient.

5. The surgical device according to claim 4, wherein the suction arrangement is controlled by a user.

6. The surgical device according to claim 1, further comprising:
a sterile irrigation system configured to deliver a fluid to an area adjacent to the surgical tip wherein the irrigation system is independently positionable within the patient and wherein the irrigation system is inserted into the body through a separate opening of the body.

7. The surgical device according to claim 6, wherein the sterile irrigation system is configured to be controlled by a user.

8. The surgical device according claim 1, wherein the tip is configured as one of a rounded tip, serrated, a chisel, a two dimensional cutting profile, an angle and a wedge.

9. A surgical device comprising:
a body defining an interior volume, wherein the body has a first end and a second end, wherein the first end and the second end are hollow;
at least one optical fiber configured to illuminate an area inside a patient near the second end of the body;
a surgical tip configured to fit through the first end and the second end of the body and into a patient in an endoscopic incision to a spinal area to impart a vibration to a contacted object within the patient;
a piezoelectric arrangement configured to initiate a piezoelectric effect to actuate a vibration in the surgical tip,
at least one optical fiber to receive video images near the surgical tip;
at least one display device connected to the optical fiber such that a surgeon may visually see actions of the surgical tip during operation; and
a sterile irrigation system configured to impart a fluid to an area near the surgical tip during operation.

10. The surgical device according to claim 9, further comprising:
at least one recording device connected to the at least one display device to record activities during the operation.

11. The surgical device according to claim 9, wherein at least one of the sterile irrigation system and the suction system are at least one of user controlled and computer controlled.

12. The surgical device according to claim 9, wherein the piezoelectric arrangement is configured such that a user may select a vibration frequency of the arrangement.

13. The surgical device according to claim 9, wherein the tip is configured to cut at least one of bone and soft tissue.

14. The surgical device according to claim 9, wherein the tip is configured as one of a rounded tip, a hook, serrated, a chisel, a wedge and a burr.

15. The surgical device according to claim 9, wherein the tip is configured to be placed in an incision less than 1 inch in length.

16. The surgical device according to claim 9 wherein the irrigation system is configured with a suction system configured to remove at least one of fluid and objects from the endoscopic incision.

17. A method, comprising:
making an incision into a patient spinal area through a robotic surgical device;
inserting an endoscopic piezoelectric device into the incision through use of the robotic surgical device;
performing at least one function using a piezoelectric effect with the piezoelectric device on the patient with the robotic surgical device; and
closing the incision.

18. The method according to claim 17, further comprising:
varying at least one value of the piezoelectric device for the at least one function.

19. The method according to claim 18, wherein the value is a frequency of vibration of a surgical tip of the piezoelectric device.

20. The method according to claim 17, wherein the function is a cutting of bone with a surgical tip of the piezoelectric device.

21. The method according to claim 17, further comprising:
inserting a retraction device into the incision in the patient spinal area prior to inserting the endoscopic piezoelectric device into the incision.
